(19) United States
(12) Patent Application Publication
(10) Pub. No.: US 2015/0184111 A1
(43) Pub. Date: Jul. 2, 2015

(54) Perfume and use thereof

(71) Applicant: Jose A. Rodriguez, Burbank, CA (US)

(72) Inventor: Jose A. Rodriguez, Burbank, CA (US)

(21) Appl. No.: 13/998,934

(22) Filed: Dec. 26, 2013

(51) Int. Cl. C11B 9/00; A61K 8/92; B65D 85/00; A61Q 19/10

(52) U.S. Cl. CPC C11B 9/00; A61Q 19/10; A61K 8/922; B65D 85/70

(57) ABSTRACT

A perfume or fragrance composition having a mixture of components, by weight, consisting of:
a) 2 to 4 parts fir needle
b) about 1 part eucalyptus
c) about ½ part gardenia.

### BACKGROUND OF THE INVENTION

This invention relates generally to dispersible fragrances or perfume compositions, and more generally and superior such compositions.

There is need for superior perfumes or fragrances, and more particularly combinations of essential oils producing such superior fragrances.

### SUMMARY OF THE INVENTION

It is a major object of the invention to provide dispersible and new fragrances as in dispersible form, and characterized by a combination of fir needle, eucalyptus, and gardenia fragrances. Preferably the fragrances are present in combination, by weight, as follows:
2 to 4 parts fir needle
about 1 part eucalyptus,
and, about ½ part gardenia.

Such components are typically in the form of essential oils, as for example in plant extract form. Also, the oil components are typically dispersed into an aqueous phase, for use, as from spray, lotion, or other containers.

These and other objects and advantages of the invention, as well as the details of an illustrative embodiment, will be more fully understood from the following specification and drawings, in which:

### DRAWING DESCRIPTION

FIG. 1 shows a spray container, from which the perfume is to be dispersed;

FIG. 2 shows a container for the product, and to be applied manually, as in cream form;

FIG. 3 shows a lotion container; and

FIG. 4 shows scenting of soap.

### DETAILED DESCRIPTION

As referred to, three components of the fragrance composition consists, by weight of
2-4 parts fir needle
about 1 part eucalyptus
about ½ part gardenia.

The components are a essential oil, typically extract form, suitable for dispersion. Dispersing agents are typically water, or alcohol (ethyl), in liquid form.

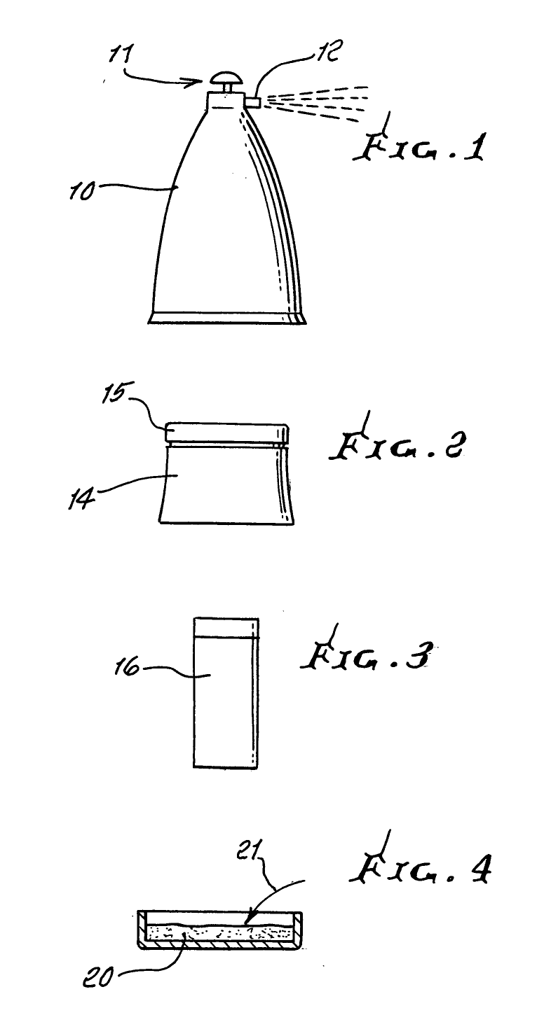

FIG. 1 shows a spray container 10 for the mixture of dispersible components. A plunger 11 is manually movable downwardly to effect spray disperse from an outlet spout 12. FIG. 2 shows a jar 14 having a lid 15 that is openable for access to the dispersible composition. FIG. 3 shows a lotion container 16, for the composition. FIG. 4 shows use of the perfume, as for scenting soap 20. See application 21 of dispersed liquid.

I claim:

1. A perfume or fragrance composition having a mixture of components, by weight, consisting of:
a) 2 to 4 parts fir needle
b) about 1 part eucalyptus
c) about ½ part gardenia.

2. The composition of claim 1 wherein said fir needle consist of about 4 parts.

3. The composition of claim 1 wherein at least one of the components is in essential oil form.

4. The composition of claim 1 wherein all of the components are in mixed essential oil form.

5. The composition of claim 1 wherein the components are dispersed in water.

6. The composition of claim 1 including one of the following in which the component mixture is contained:
i) spray container
ii) jar
iii) lotion container.

7. The composition of claim 5 including one of the following in which the component mixture is contained:
i) spray container
ii) jar
iii) lotion container.

8. The composition of claim 7 wherein all of the components are in mixed essential oil form.

9. The composition of claim 1 wherein all of the components are in extract form.

10. The composition of claim 8 wherein all of the components are in extract form.

11. The method that includes combining the mixture of components as defined in claim 1, and dispersing said mixture in liquid form.

12. The method of claim 11 wherein the mixture is dispersed into soap.
